(19) United States
(12) Patent Application Publication
(10) Pub. No.: US 2007/0043370 A1
(43) Pub. Date: Feb. 22, 2007

(54) Callus elongating/regenerating device

(76) Inventors: Minoru Ueda, Aichi (JP); Hideharu Hibi, Aichi (JP)

Correspondence Address: MORGAN LEWIS & BOCKIUS LLP, 1111 PENNSYLVANIA AVENUE NW, WASHINGTON, DC, 20004, US

(21) Appl. No.: 10/555,812

(22) PCT Filed: Apr. 30, 2004
(86) PCT No.: PCT/JP04/06359

§ 371 Date: Jul. 25, 2006

(30) Foreign Application Priority Data
May 7, 2003 (JP) 2003-129342

(51) Int. Cl. A61F 2/30

(52) U.S. Cl. 606/71

(57) ABSTRACT

In a callus elongating/regenerative device (1), a plate (3) is provided in a bar-shaped fashion to move in a three-dimensional direction depending on a pre-mutilated bone shape so as to bridge a mutilated bone portion (2). A bone cut end (8) is severed to produce two bone pieces (4) which are held by brackets (5). The brackets (5) are pulled to move along the plate (3) to three-dimensionally extend a callus (9) osteoplastically formed between a severed bone end (10) and a severed end surface (11). Since the brackets (5) are formed to move while surrounding the plate (3), the sizes of the plate (3) and the brackets (5) are such a degree as to be placed in a wound to-be osteoplastically treated. Thus, the callus (9) can be three-dimensionally extended with a most part of the callus elongating/regenerative device (1) retained in the wound.

### FIELD OF THE INVENTION

The present invention relates to a callus elongating/regenerative device for use in orthopedics and oral surgery.

### BACKGROUND OF THE INVENTION

Among orthopedic surgeries, a remedial operation has been made to partly ablating a bone to clinically treat a tumor, a cyst,.an injury or a jawbone deformation (e.g., bone dysplasia) appeared in the bone. The operation develops a mutilated bone portion, to which a bio-artifact is applied as a replacement with the use of a plate 100 as shown in FIG. 17. Otherwise, an auto bone graft has been made in which a self bone 101 is taken from other part of the subject body so that the self bone 101 is transplanted into the mutilated bone portion.

Using the replacement does not always insure a sufficient bio-compatibility with a subject body to be surgically treated. The auto bone graft accompanies a surgical attack against a normal part of the body.

In order to obviate these inconveniences, a callus extension method has been advocated to regenerate a new bone at the mutilated bone portion by osteoplastically extending the callus to fill the mutilated bone portion. In the callus extension method, the bone piece is applied to the bone cut end 102 to gradually extend the callus formed between the bone piece and the bone cut end 102 depending on the degree how the callus grows therebetween.

Among the devices to develop the callus, a threaded portion is provided on a bridge member extending over the mutilated bone portion as shown in Japanese Laid-open Patent Application No. 9-215699. A movable member is provided to move in unison with the bone piece so that the movable member gradually moves along the bridge member by means of a screw nut.

A rack member is formed on the bridge member and a pinion is placed on the movable member to mesh with the rack member as shown in Japanese Laid-open Patent Application No. 11-262491. The pinion is driven to gradually move the bone piece along the bridge member.

It is, however, impossible to place the device (Japanese Laid-open Patent Application No. 9-215699) in the wound, and thus involving inconveniences in daily life with the device attached to the body.

As for the device (Japanese Laid-open Patent Application No. 11-262491), it is possible to place the device in the wound since both the bridge member and the movable member are reduced in their thickness. The device, however, requires a complicated mechanism in the wound upon driving the rack and the pinion. Further, it is only possible to move the callus two-dimensionally with the use of the rack and the pinion.

Therefore, it is an object of the invention to overcome the above drawbacks, and provide a callus elongating/regenerat ive device which is capable of three-dimensionally extending a callus in the wound with a relatively simple structure.

### DISCLOSURE OF THE INVENTION

According to the invention, there is provided a callus elongating/regenerative device in which a plate is provided in a bar-shaped fashion to move in a three-dimensional direction depending on a pre-mutilated bone shape so as to bridge a mutilated bone portion. A bracket has a slide portion movably provided while surrounding the plate. The bracket holds a bone piece located at the mutilated bone portion formed between both ends of a bone cut end. The bone piece extends a callus from the bone cut end and movably arranged along a lengthwise direction of the plate. A wire is connected to the bracket so that the bracket moves in the lengthwise direction of the plate so as to osteoplastically extend the callus.

Upon operating the wire with the bracket connected to one end of the wire, it becomes possible to osteoplastically extend the callus in the three-dimensional direction.

Since it suffices that the bracket moves while surrounding the plate, the dimensional sizes of the bracket and the plate are such as to be placed within the wound.

This makes it possible to accommodate most parts of the device within the wound only except for an operating member, whereby enabling an operator to extend the callus in the three-dimensional direction with the most parts of the device accommodated within the wound. This also enables a patient to lead the life without inviting inconveniences in daily basis.

Upon treating the mutilated bone portion of a lower jawbone, the situation enables the patient to put the operating member behind the ear without disturbing the outer appearance.

Since the bracket is operated by the wire, it is possible to render the device into such a simple structure as to have the bracket and the plate.

Because the bracket and the plate are discrete individual members, it is possible to optionally select one of two cases. The first case is that the bracket and the plate are simultaneously attached. The second case is that only the plate is primarily attached and the bracket is later attached upon insuring the positive recovery of the wound.

It is preferable to secondarily attach the bracket from the reason that if the condition relapses with the bracket and the plate concurrently attached, it will affect on the subsequent remedial procedures. It will be left to a discretion of the operator as to whether the bracket is attached at the first stage or second stage depending on the recovery of the wound.

According to other aspect of the invention, the bracket holds a bone piece located at the mutilated bone portion formed between both ends of a bone cut end, and movably provided along a lengthwise direction of the plate. The bone cut end is severed to produce the bone piece.

This makes it possible to form the bone piece to osteoplastically extend the callus without inviting the surgical attack against the normal part of the body.

According to other aspect of the invention, a traction mechanism is provided to pull the wire by a tractive force.

This enables the operator to withdraw the wire out of the wound in accompany with the extention of the callus so as to mitigate the physical burden and the microbial contagion to which the patient owes, as opposed to the case in which the wire is pushed into the wound.

According to other aspect of the invention, the number of the brackets and the wires is two, whereby the callus is osteoplastically extended from two bone cut ends with the bone piece held by the bracket.

This enables the operator to extend the callus from the two bone cut ends so as to osteoplastically fill the mutilated bone portions two times as quickly as the case in which the callus develops only from one bone cut end.

According to other aspect of the invention, one of the two brackets has a passage hole to pass the wire therethrough so as to move the other bracket. The other bracket has a passage hole to pass the wire therethrough so as to move the former bracket.

Such is the structure that it holds the wire along the plate without departing from the proximity of the plate.

According to other aspect of the invention, the plate has a groove to accommodate the wire in the lengthwise direction of the plate. This makes it possible to effectively prevent the wire from falling off the plate.

According to other aspect of the invention, the traction mechanism has a rod connected to the wire. A cylindrical sleeve is provided to movably hold the rod in its axial direction with the sleeve fixedly positioned against the plate. A stress concentration portion is provided to concentrate a bending stress on the sleeve when the bending moment is applied to the sleeve.

Such is the structure that it preferentially bends the sleeve at the stress concentration portion to avoid the traction mechanism from three-dimensionally interferring with other part of the body.

According to other aspect of the invention, the traction mechanism has a rod connected to the wire. A cylindrical sleeve is provided to movably hold the rod in its axial direction with the sleeve fixedly positioned against the plate. A graduated portion is provided with the rod to indicate its lengthwise dimension when the rod is protracted out of the sleeve.

With the graduated portion thus provided, it is possible to visually recognize how far the bracket is pulled by the tractive force of the wire (i.e., a displacement of the bracket). This enables the operator to readily measure how far the callus is osteoplastically extended (i.e., an extension of the callus).

According to other aspect of the invention, the traction mechanism has rod connected to the wire. A cylindrical sleeve is provided to movably hold the rod in its axial direction with the sleeve fixedly positioned against the plate. A tension measuring member is provided to measure a tension applied to the wire when the bracket is pulled by the tractive force.

This enables the operator to adjust the displacement of the bracket depending on the tension applied to the wire, thus protecting the callus against an excessive burden upon extending the callus.

According to other aspect of the invention, the traction mechanism has a rod connected to the wire, and having a male-threaded portion on an outer surface of the rod. A cylindrical sleeve is provided to movably hold the rod in its axial direction with the sleeve fixedly positioned against the plate. An adjustment member has a female-threaded portion into which the male-threaded portion is driven, and the female-threaded portion is driven to move along the rod so as to adjust a displacement of the bracket pulled by the wire.

Such is the structure that it moves the rod stably without departing from the axial direction, thus enabling the operator to stably pull the bracket by the tractive force.

According to other aspect of the invention, the wire is held at a hollow space appeared between an inner wall of the sleeve and an outer wall of the rod. The wire is adapted to removably engage with an end surface of the rod.

This makes it possible to insure an easy connection and disconnection between the wire and the rod, while at the same time, dimensionally reducing the lengths of the rod and sleeve.

When the rod and the wire are unremovably connected, it requires for the rod and the wire to have lengths corresponding to an entire extension of the callus.

Under the condition that the wire is removably provided against the rod, if the rod moves excessively beyond the sleeve in accompany with the extension of the callus, the structure enables the operator to disconnect between the rod and the wire so as to adjust the protractive displacement of the rod without adjusting the wire. This makes it possible to determine lengths of the rod and the sleeve regardless of the entire extension of the callus.

### BRIEF DESCRIPTION OF THE DRAWINGS

Preferred forms of the present invention are illustrated in the accompanying drawings in which:

FIG. 1 is a perspective view of a callus elongating/regenerative device according to a first embodiment of the invention;

FIG. 2 is a longitudinal cross sectional view of a bracket of the callus elongating/regenerative device;

FIG. 3 is a longitudinal cross sectional view of the callus elongating/regenerative device attached to a bone cut end;

FIG. 4 is a latitudinal cross sectional view taken along the line IV—IV of FIG. 3;

FIG. 5 is a plane view of the callus elongating/regenera tive device;

FIG. 6 is a longitudinal cross sectional view of the callus elongating/regenerative device in which the callus is formed;

FIG. 7 is a perspective view of the callus elongating/regenerative device depicted to show that the callus is osteoplastically growing;

FIG. 8 is a perspective view of the callus elongating/regenerative device depicted to show that the callus has been osteoplastically formed;

FIG. 9 is a perspective view of the callus formed between ablated end surfaces appeared upon removing a tumor-afflicted bone;

FIG. 10 is a longitudinal cross sectional view of an operating member of the callus elongating/regenerative device according to a second embodiment of the invention;

FIG. 11 is a latitudinal corss sectional view taken along the line XI—XI of FIG. 10;

FIG. 12 is a perspective view of the operating member which is adjustably reduced at its length;

FIG. 13 is a longitudinal cross sectional view of a callus elongating/regenerative device according to a third embodiment of the invention;

FIG. 14 is a perspective view of a callus elongating/regenerative device depicted to show that the sleeve is bent at a stress concentration portion according to a fourth embodiment of the invention;

FIG. 15 is an exploded perspective view of the operating member;

FIG. 16 is a perspective view of a callus elongating/regenerative device according to a fifth embodiment of the invention; and

FIG. 17 is a prior art device used to fill a mutilated bone portion with a callus.

### BEST MODES TO CARRY OUT THE EMBODIMENTS OF THE INVENTION

### FIRST EMBODIMENT OF THE INVENTION

In the following description of the depicted embodiments, the same reference numerals are used for features of the same type.

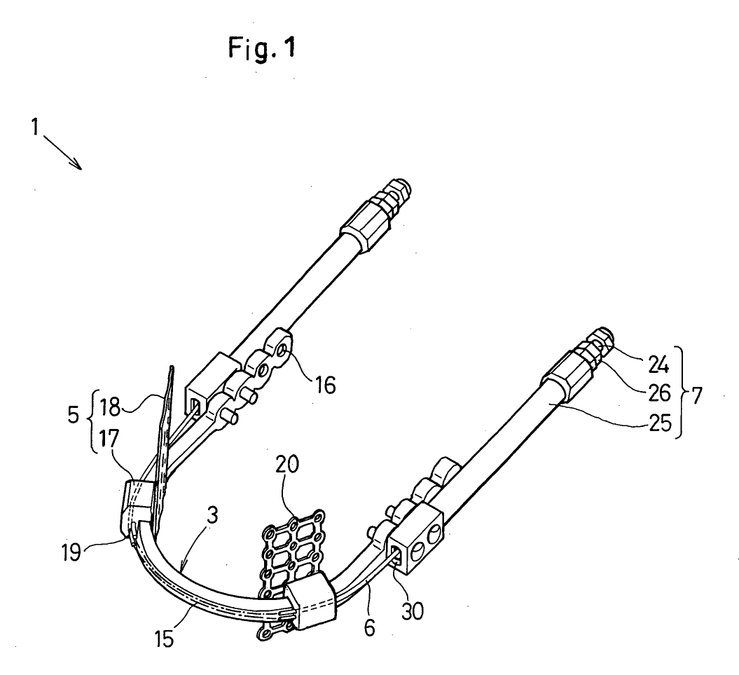

Referring to FIGS. 1 through 6 which shows a callus elongating/regenerative device 1 (referred to merely as“device 1” at the latter part of the description) according to a first embodiment of the invention, the callus elongating/regenerative device 1 has a plate 3 which bridges a mutilated bone portion 2, and further having two brackets 5 provided to slidably hold a bone piece 4 along the lengthwise direction of the plate 3. To each of the brackets 5, a wire 6 is connected to move the brackets 5 along the lengthwise direction of the plate 3. A traction mechanism is provided to operate the wire 6 as an operating member 7.

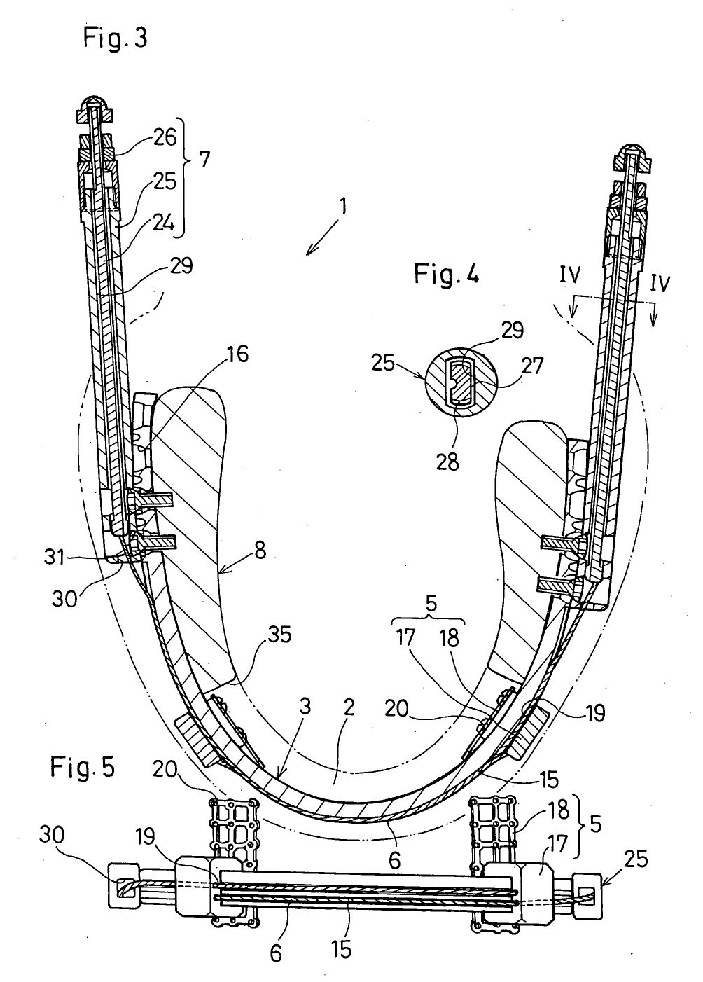

The mutilated bone portion 2 is a space appeared between right and left bone cut ends 8 by partly ablating a bone at the time of clinically treating a tumor, a cyst, an injury or a jawbone deformation developed in the bone as shown in FIG. 3.

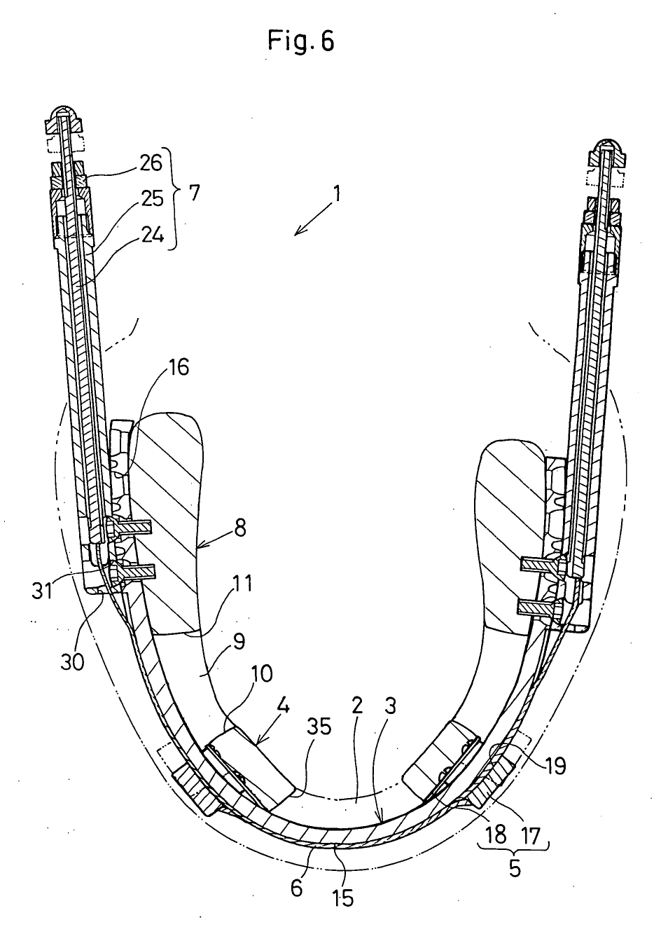

The bone piece 4 is an osteoplastic block provided to move as it forms a callus 9 as shown in FIG. 6. By way of illustration, the bone piece 4 is prepared by severing the bone cut end 8 at a predetermined length. This is arranged to osteoplastically form and extend the callus 9 between a severed end surface (as referred to as “severed end surface 11” hereinafter) of the bone cut end 8 and a severed end surface (as referred to as “severed bone end 10” hereinafter) of the bone piece 4.

The plate 3 forms a bar-shaped configuration and three-dimensionally changes its shape in accordance with the pre-mutilated bone shape. Material of the plate 3 can be selected among a pure titanium (JISH4670), a titanium-based alloy{Ti-6Al-4V (JISH4657), ASTM F-136, Ti-6l-4V ELI} and a stainless steel {SUS304, SUS 316 (JISG4304)}.

Since the plate 3 is placed in a wound to be osteoplastically treated, it it preferable to select the pure titanium or the titanium-based alloy in consideration of biocompatibility. With the use of a well-known bender, it is possible to deform the plate 3 into the bar-shaped configuration from a linear column element.

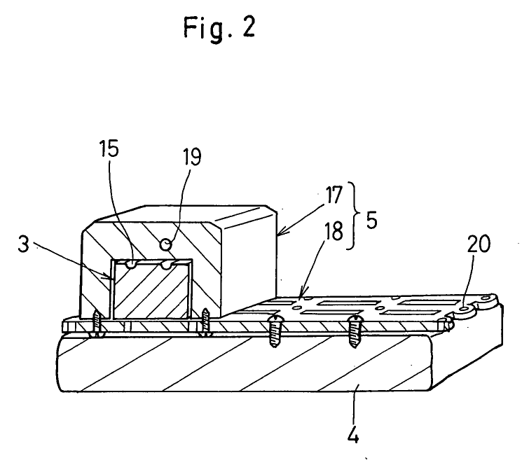

As shown in FIGS. 2 and 5, the plate 3 has two rows of longitudinal grooves 15 to accommodate the right and left side wires 6 in its lengthwise direction. With the right and left ends of the plate 3, an array of holes 16 is provided to fasten the plate 3 and the operating member 7 to the bone cut end 8 by means of a pin or a screw as shown in FIGS. 1 and 3. Four holes 16 are arranged on the plate 3 in its lengthwise direction by way of illustration. Among the four holes 16, appropriate ones can be selected depending on the size and shape of the bone to be osteoplastically extended.

As shown in FIGS. 3 and 6, the brackets 5 is angularly shaped in cross section, and having right and left sliders 17 provided to move with its inner portion surrounding the plate 3.

In addition to the sliders 17, each of the brackets 5 has a holder 18 provided to hold the bone piece 4. To one slider 17, one end of the corresponding wire 6 is removably connected. The slider 17 has a passage hole 19 provided to pass another wire 6 therethrough.

As for the connection between the slider 17 and the wire 6, one end of the wire 6 may have a male-threaded portion (not shown) which is driven into a female-threaded portion (not shown) formed on the slider 17. Alternatively, one end of the wire 6 may have a concave portion which fits into a convex portion formed on the slider 17.

The holder 18 of the bracket 5 forms a lattice-work pad and having a screw hole 20 to hold the bone piece 4 by means of a screw as shown in FIGS. 1 and 5. The holder 18 is adjustably cut depending on the size and shape of the bone piece 4. The bone piece 4 is placed on the holder 18 and fastened to the holder 18 by means of the screw as shown in FIG. 2. The slider 17 and the holder 18 are fixed together with the plate 3 interposed therebetween so as to constitute the bracket 5. The slider 17 and the holder 18 are made of the same material as used to form the plate 3. Considering that the slider 17 and the holder 18 are placed in the wound, it is preferable to use the pure titanium or the titanium-based alloy as their material.

The wire 6 is connected at one end to the slider 17 and other end to one end of a rod 24 of the operating member 7. As for the connection between the wire 6 and the rod 24, one end of the wire 6 may have a male-threaded portion which is driven into a female-threaded portion formed on the rod 24. Alternatively, one end of the wire 6 may have a concave portion which fits into a convex portion formed on the rod 24. The wire 6 is partly placed in the slider 17 through the passage hole 19, and is partly accommodated into the longitudinal groove 15.

Upon operating the rod 24 by pulling the wire 6 with a tractive force, the tractive force moves the bracket 5 along the plate 3 with the bone piece 4 held by the bracket 5.

Material of the wire 6 can be selected as a metallic cord among a pure titanium (JISH4670), a titanium-based alloy {Ti-6Al-4V (JISH4657), ASTM F-136, Ti-6Al-4V ELI } and a stainless steel {SUS304, SUS 316 (JISG4304)}. Material of the wire 6 may be synthetics such as a nylon-based polymer or a polylactic acid polymer. Considering that the wire 6 is placed in the wound, it is preferable to use the pure titanium or the titanium-based alloy as the material. The wire 6 may be a single line wire or helically stranded wire.

The operating member 7 has the rod 24 connected at one end to the wire 6, and having a sleeve 25 into which the rod is movably accommodated as shown in FIGS. 3 and 6. The rod 24 has double nuts 26 provided to adjustably move the rod 24 in relative to the sleeve 25. The rod 24 has opposed flat surfaces 27 in the lengthwise direction, and further having opposed male-threaded portions 28 as shown in FIG. 4.

The sleeve 25 is in a cylindrical configuration with its inner space as a hollow portion 29. The cross section of the sleeve 25 corresponds to that of the rod 24 so as to prevent the rod from rotating around its axial elongation. The sleeve 25 has an exit opening 30 provided to introduce the wire 6 ouside from one end of the rod 24. The sleeve 25 has an array of holes 31 at one side to fasten the operating member 7 to the bone cut end 8 together with the plate 3 as shown in FIGS. 3 and 6. This makes it possible to fix the sleeve 25 at its position against the plate 3. With the two holes 31 longitudinally arranged, it is possible to fasten the sleeve 25 and the plate 3 firmly to the bone cut end 8, as opposed against the case in which only one hole 31 is provided.

The double nuts 26 constitutes two hexagonal nuts fastened to the other end of the rod 24. The double nuts 26 moves along the rod 24 to adjust the displacement of the bracket 5 so as to serve as an adjustment tool. The rod 24, the sleeve 25 and the double nuts 26 can be made of the same material as used for the plate 3, the bracket 5 and the wire 6. Considering that the operating member 7 is often placed ouside the wound, it is not always necessary to care about the biocompatibility so much as the plate 3, the bracket 5 and the wire 6. It is preferable to use the stainless steel as the material of the operating member 7 from a cost-saving point of view.

### How Attach the Callus Elongating/Regenerative Device

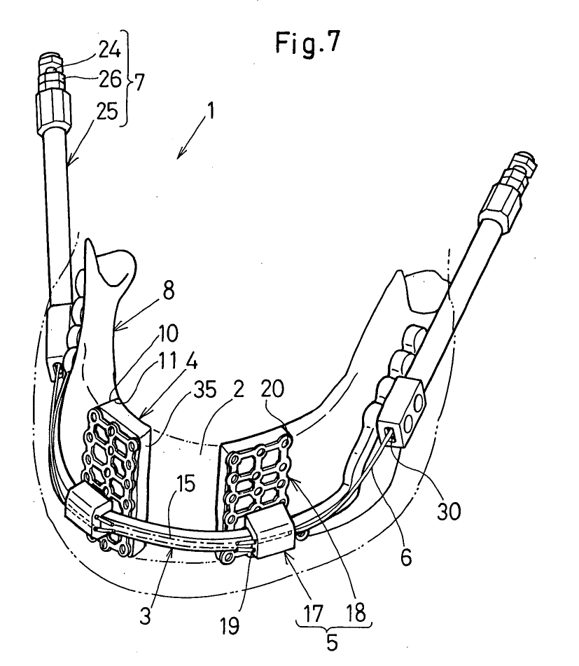

In the first embodiment of the invention, the callus elongating/regenerative device 1 is exemplarily applied to the mutilated bone portion 2 appeared upon removing the tumor out of a lower jawbone as shown in FIGS. 7, 8 and 9.

The jaw is cut to expose the lower jawbone so as to clinically remove the tumor from the lower jawbone. This occasions to determine the position to fix the plate 3 in place, while at the same time, determining the bone-severing position in connection with the bone cut ends 8.

The plate 3 is attached at both ends to the corresponding bone cut ends 8 through the hole 16 by means of a pin or a screw. The sleeve 25 is attached at one end to the corresponding bone cut ends 8 through the hole 31 by means of a pin or a screw so as to bridge the mutilated bone portion 2. The plate 3 has been three-dimensionally deformed in accordance with the configuration of the pre-mutilated jawbone before removing the tumor.

Then, the bracket 5 is attached to the plate 3 with the holder 18 located inside the plate 3 and with the slider 17 surrounding the plate 3. The slider 17 and the holder 18 are fastened through the screw hole 20 by means of the screw so as to attach the bracket 5 to the plate 3. In this instance, the holder 18 has been adjusted at its dimension depending on the size and shape of the bone piece 4.

The wire 6 is attached to the plate 3, the slider 17 and the rod 24. Namely, the wire 6 is connected at one end to the corresponding slider 17, and concurrently brought to pass through the passage hole 19 of another slider 17. The wire 6 is connected at the other end through the exit opening 30 to one end of the rod 24 protracted from the other end of the sleeve 25. The operating member 7 brings one end of the rod 24 inside the hollow portion 29 of the sleeve 25 to fasten the double nuts 26 to the male-threaded portion 28 of the rod 24. In this way, the callus elongating/regenerative device 1 is completed at its attachment to the lower jawbone as shown in FIG. 3.

### Operation of the Callus Elongationg/Rerenerative Device

The following are description to show how the callus elongating/regenerative device 1 is operated to form the callus 9. At the bone-severing position of the bone cut ends 8, the bone cut ends 8 are severed to produce the bone pieces 4 to fasten the bone pieces 4 to the holder 18 through the screw hole 20 by means of the screw. This holds the bone pieces 4 with the severed end surface 11 abutted against the severed bone end 10 which appears upon producing the bone pieces 4 as shown in FIG. 7.

In accompany with the callus 9 formed between the severed end surface 11 and the severed bone end 10, the wire 6 is manipulatively pulled to slide the bracket 5 along the plate 3 in such a direction as to extend the callus in the lengthwise direction.

In this instance, the tractive force appears on the wire 6 upon adjustably driving the double nuts 26 to move it along the male-threaded portion 28 of the rod 24 to protract the rod 24 from the rear end of the sleeve 25. Namely, adjusting the double nuts 26 pulls the rod 24 out of the sleeve 25 with the double nuts 26 engaged against the rear end surface of the sleeve 25. This gives the wire 6 the tractive force to pull the bracket 5 along the plate 3 so as to extend the callus 9 in such a direction as the bracket 5 moves.

As for a regenerative extension speed of the callus 9, the callus 9 usually grows at the speed of approx. 1 mm per a day. The tractive force pulls the rod 24 from the sleeve 25 in correspondence to the extension speed of the callus 9.

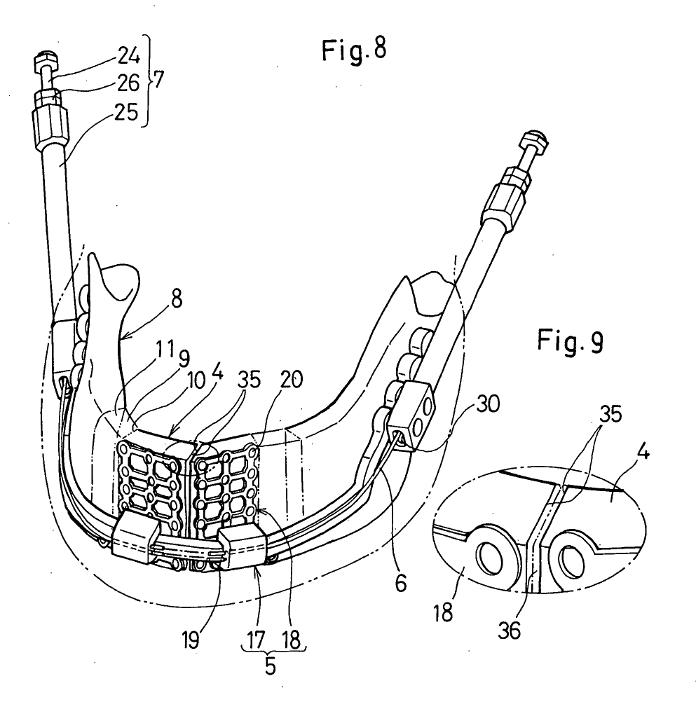

The tractive force further moves the brackets 5 to make ablated side surfaces 35 come in contact with each other as shown in FIG. 8. The ablated side surfaces 35 are side ends of the bone piece 4 appeared when the tumor is clinically removed from the jawbone. When the callus formation 36 fills up a gap between the ablated side surfaces 35 as shown in FIG. 9, the jaw is clinically cut again to take off the callus elongating/regenerative device 1 from the jawbone.

### Advantages of the Invention

As aforementioned, the clinical operation bridges the mutilated bone portion 2 by the plate 3 shaped three-dimensionally in correspondence to the bone shape to be osteoplastically treated. Then, the bone cut ends 8 are severed at both sides to produce the two bone pieces 4 which are attached to the corresponding brackets 5 so as to osteoplastically extend the callus 9 formed between the severed bone end 10 and the severed end surface 11 in. accompany with the pulling operation of the wire 6.

With the use of the operating member 7 upon pulling the wire 6, it is possible to osteoplastically extend the callus 9 along the three-dimensional direction in accordance with the plate 3 three-dimensionally shaped in correspondence to the bone shape.

With the bone pieces 4 produced from the bone cut ends 8 and with callus formed between the severed bone end 10 and the severed end surface 11 which appear at the time of producing the bone pieces 4, it is possible to prepare the bone pieces 4 and extendedly form the callus 9 without inviting the surgical attack against the normal portion of the body.

Since the callus elongating/regenerative device 1 enables the operator to use the bone pieces 4 in the proximity of the portion to be osteoplastically extended, it is preferable from the viewpoint of bio-compatibility.

Since it suffices that the brackets 5 move while surrounding the plate 3, the dimensional sizes of the brackets 5 and the plate 3 are such as to be placed within the wound.

This makes it possible to accommodate most parts of the device 1 within the wound only except for the operating member 7, whereby enabling an operator to extend the callus 9 in the three-dimensional direction with the most parts of the device 1 placed within the wound. This also enables a patient to lead the life without inviting inconveniences in daily basis.

Upon osteoplastically treating the mutilated bone portion 2 of the lower jawbone, the situation enables the patient to put the operating member 7 behind the ear without disturbing the outer appearance.

Since it suffices to movably design the brackets 5 while surrounding the plate 3, it is possible to render the device 1 into such a simple structure as to have the brackets 5 and the plate 3 accommodatable within the wound.

Because the brackets 5 and the plate 3 are discrete individual members, it is possible to optionally select one of the two cases as follows.

The first case is that the brackets 5 and the plate 3 are simultaneously attached. The second case is that only the plate 3 is primarily attached and the brackets 5 are later attached after observing the positive recovery of the wound.

It is preferable to secondarily attach the brackets 5 from the reason that if the condition relapses with the brackets 5 and the plate 3 concurrently attached, it will affect on the subsequent remedial procedures. It will be left to the manipulative discretion as to whether the brackets 5 are attached at the first stage or second stage depending on the observation of the recovery.

The device 1 has the traction mechanism to pull the wire 6 to move the brackets 5 along the plate 3 with the tractive force produced by the operating member 7.

This enables the operator to withdraw the wire 6 out of the wound in accompany with the extention of the callus 9 so as to mitigate the physical burden and the microbial contagion to which the patient owes, as opposed to the case in which the wire is pushed into the wound.

The two brackets 5 and the two wires 6 are used to extend the callus 9 from the corresponding bone cut ends 8 with the bone piece 4 held by the corresponding brackets 5.

This enables the operator to extend the callus 9 from the two bone cut ends 8 so as to osteoplastically fill the mutilated bone portions 2 two times as quickly as the case in which the callus 9 develops solely from one bone cut end 8.

One of the two brackets 5 has the passage hole 19 to pass the wire 6 therethrough so as to move the other bracket 5, and the other bracket 5 has the corresponding passage hole 19 to pass the wire 6 therethrough so as to move the former bracket 5.

Such is the structure that it holds the wire 6 along the wire 6 without departing from the proximity of the plate 3.

The plate 3 has the longitudinal groove 15 to accommodate the wire 6 in the lengthwise direction of the plate 3. This makes it possible to effectively prevent the wire 6 from falling off the plate 3.

The adjustment member has the double nuts 26 to move it along the male-threaded portion 28 of the rod 24 so as adjust the displacement length of the bracket 5, thus enabling the operator to stably pull the rod 24 without departing from its axial direction. This makes it possible to osteoplastically extend the callus 9 without departing the callus 9 from the axial direction of the plate 3.

### Second Embodiment of the Invention

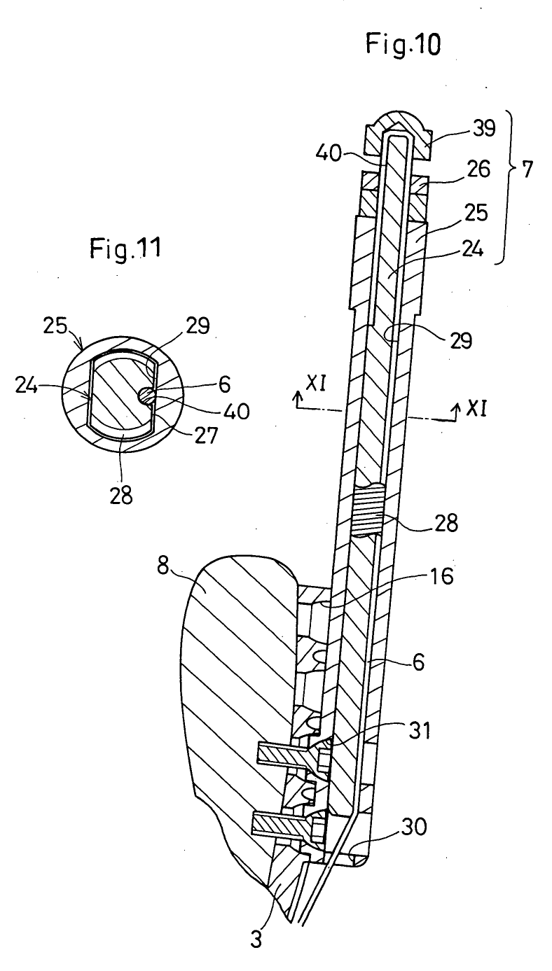

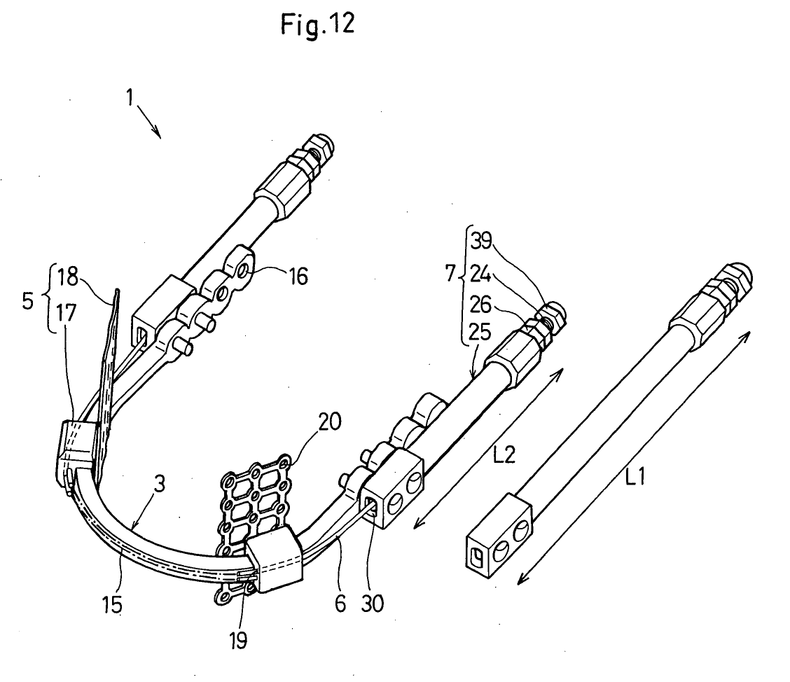

FIGS. 10 and 12 show a second embodiment of the invention in which a cap nut 39 is provided at the rear end of the rod 24 in the operating member 7. The flat surface 27 of the rod 24 has a lengthwise groove 40 to accommodate the wire 6. The groove 40 extends along the full length of the one flat surface 27 to a part of the other flat surface 27 via the rear end tip of the rod 24. This holds the wire 6 in its axial direction within a hollow space 29 between an inner wall of the sleeve 25 and an outer wall of the rod 24.

In this instance, the cap nut 39 removably secures the halfway portion of the wire 6 bent along the rear end tip of the rod 24.

### Advantages of the Invention

With the wire 6 held within the hollow space 29 of the sleeve 25 so as to removably engage against the rear end tip of the rod 24 by means of the cap nut 39, it is possible to insure an easy connection and disconnection between the wire 6 and the rod 24, while at the same time, reducing the lengths of the rod 24 and sleeve 25.

When the rod 24 and the wire 6 are unremovably connected, it requires for the rod 24 and the wire 6 to have a length corresponding to an entire extension of the callus 9.

Under the condition that the wire 6 is removably provided against the rod 24, if the rod 24 moves excessively beyond the sleeve 25 in accompany with the extension of the callus 9, the structure enables the operator to disconnect between the rod 24 and the wire 6 so as to adjust the protractive displacement of the rod 24 without adjusting the wire 6. This makes it possible to determine lengths of the rod 24 and the sleeve 25 regardless of the entire extension of the callus 9. The arrangement dimensionally reduces the sleeve 25 from the length L1 to the length L2 as illustrated in FIG. 12.

### Third Embodiment of the Invention

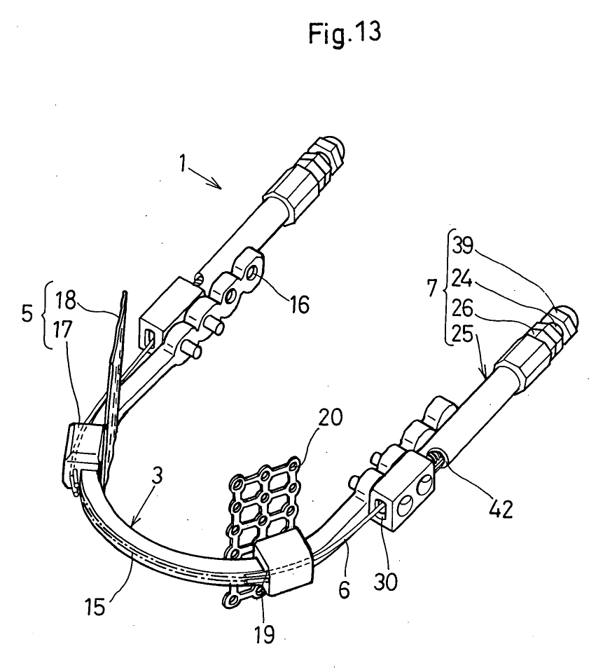

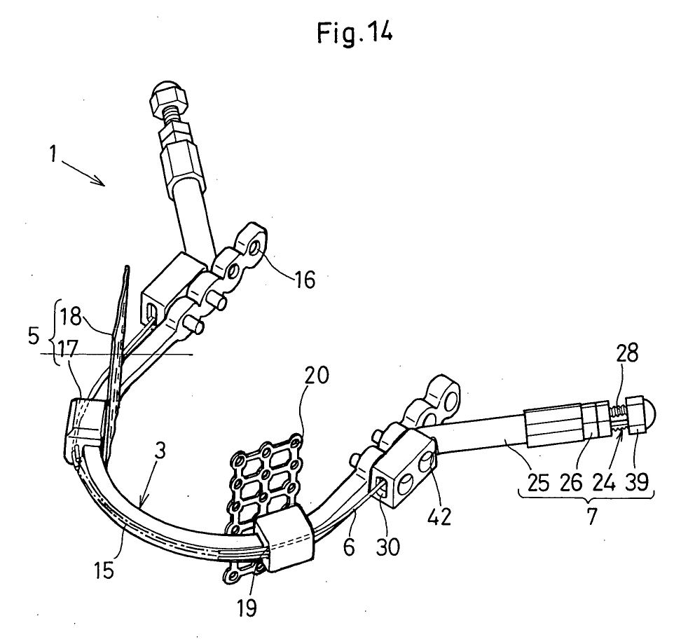

FIGS. 13 and 14 show a third embodiment of the invention in which a stress concentration portion 42 is provided on the sleeve 25 so that a concentrated stress develops at the stress concentration portion 42 when a bending moment is applied to the sleeve 25. The stress concentration portion 42 is formed as a notched portion at the side of the sleeve 25 in which the exit opening 30 resides.

The notched portion is formed by cutting an outer surface of the sleeve 25 in the manner to expose a notched side to a portion opposite to the plate 3. With the use of the well known bender tool, it is possible to bent the sleeve 25 at the the stress concentration portion 42 in such a direction as moving away from the plate 3.

### Advantages of the Invention

Such is the structure that it preferentially bends the sleeve 25 at the stress concentration portion 42 to avoid the operating member 7 from three-dimensionally interferring with other part of the body.

### Fourth Embodiment of the Invention

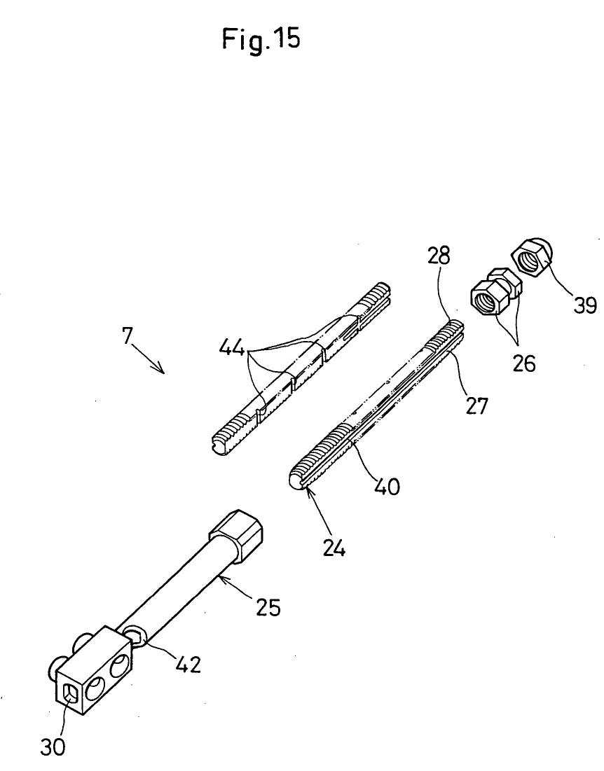

FIG. 15 show a fourth embodiment of the invention in which a graduated portion 44 is provided with the rod 24 in order to indicate its lengthwise dimension when the rod 24 is protracted out of the sleeve 25

### Advantages of the Invention

With the graduated portion 44 thus provided, it is possible to visually recognize how far the bracket 5 is pulled by the tractive force of the wire 6 (i.e., a displacement of the bracket). This enables the operator to readily measure how far the callus 9 is osteoplastically extended (i.e., an extension of the callus).

### Fifth Embodiment of the Invention

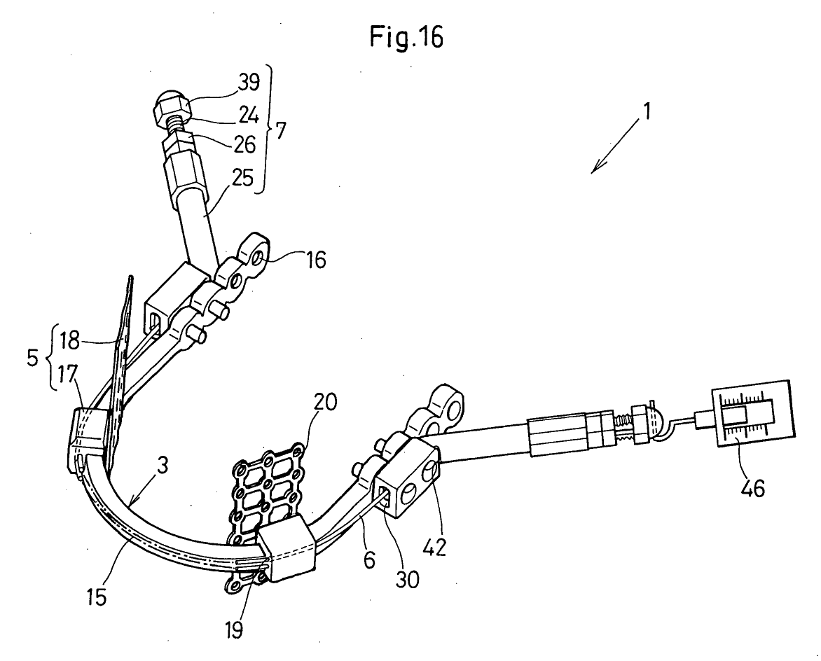

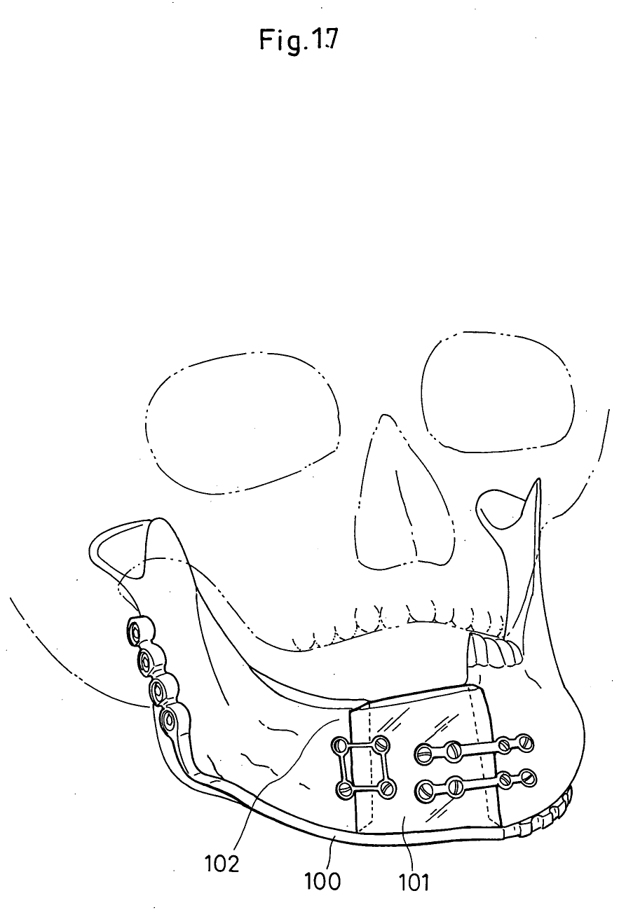

FIG. 16 show a fifth embodiment of the invention in which a tension measuring member 46 is provided as a tensiometer to measure a tension applied to the wires 6 when the brackets 5 are pulled by the tractive force. The tension measuring member 46 serves as a spring balance by way of example. The tension measuring member 46 has a hook portion connected to the cap nut 39 through its engagement hole 47. Upon moving the tension measuring member 46 to pull the wires 6, it occasions to stretch the wires 6 to develop the tension on the wires 6 at the time of osteoplastically extending the callus 9.

### Advantages

This enables the operator to adjust the displacement of the brackets 5 depending on the tension applied to the wires 6, thus protecting the callus 9 against an excessive burden upon extending the callus 9.

### Modification Forms

(a) The number of the brackets 5, wires 6 and the operating members 7 may be one each instead of two. This reduces the number of component parts to be placed within the wound so as to mitigate the burden against the body, although it occasions to extend the callus 9 from one side of the bone cut end 8.
(b) Upon bridging the mutilated bone portion 2, a two-dimensionally deformable plate may be used instead of the three-dimensionally deformable plate 3 depending on the situation.
(c) Instead of pulling the wires 6 out of the wound upon osteoplastically extending the callus 9, the wires 6 may be pushed into the wound upon extending the same.
(d) In lieu of providing the-passage hole 19 on the brackets 5 to pass the wires 6 therethrough, the wires 6 may be sandwiched between the brackets 5 and the plate 3 to produce a simple structure placed in the wound.
(e) In place of providing the groove 15 on the plate 3 to accommodate the wires 6, the wires 6 may be directly placed on an outer surface of the plate 3 to be in contact therewith.
(f) In lieu of the lower jawbone, the callus elongating/regenerative device 1 may be used to fill the mutilated bone portion 2 of other part of the body (e.g., upper jawbone) upon osteoplastically extending the callus 9 and the callus formation 36.
(g) Instead of removing the tumor, the cyst, the injury, the jaw or the jawbone deformation may be removed upon using the callus elongating/regenerative device 1.
(h) Instead of placing the operating members 7 at both sides of the plate 3, the operating members 7 may be located in different places depending on the position in which the callus elongating/regenerative device 1 is -installed. By way of example, the operating members 7 may be placed in the middle of the plate 3.
(i) Instead of severing the bone cut end 8 to produce the bone piece 4, other parts except for the bone cut end 8 may be- severed. This instance occasions to extend the callus 9 between the bone piece and the ablated side surface 35, the latter of which appears on the bone cut end 8 when the tumor is clinically removed from the jawbone.
(j) Upon using callus elongating/regenerative device 1, osteoplastic induction substances or myeloblast cells may be injected at an interface between the bone cut end 8 and the bone piece 4 at the time of extending the callus 9. This instance enables the operator to promote filling the mutilated bone portion 2 in accompany with the callus extension.
(k) Instead of providing the male-threaded portion 28 through the entire length of the rod 24, the male-threaded portion 28 may be formed only on the rear end portion of the rod 24.
(l) Instead of providing the stress concentration portion 42 in the form of the notched portion, the stress concentration portion 42 may be an uncontinuous portion made of different material, the strength of which is weaker than that of the sleeve 25. Alternatively, the stress concentration portion 42 may be a thickness-reduced portion, the thickness of which is smaller than that of the rest of the sleeve 25.
(m) Instead of providing the cap nut 39 with the engagement hole 47, the wire 6 may be connected to the tension measuring member 46 after separating the wire 6 from the rod 24 with the cap nut 39 taken off the rod 24. Alternatively, a discrete member may be secured at a rod portion between the double nuts 26 and the cap nut 39 to connect the tension measuring member 46 to the rod portion.

### INDUSTRIAL APPLICABILITY

The callus elongating/regenerative device is efficiently useful in osteoplastically extending the callus within the mutilated bone portion upon clinically treating a tumor, a cyst, an injury or a jawbone deformation developed in the bone. This enables the operator to three-dimensionally extend the callus within the wound with a relatively simple structure.

What is claimed is:

1. A callus. elongating/regenerative device comprising:
a plate provided in a bar-shaped fashion to deform in a three-dimensional direction depending on a pre-mutilated bone shape so as to bridge a mutilated bone portion;
a bracket having a slide portion provided to move while surrounding said plate, said bracket holding a bone piece located at said mutilated bone portion formed between both ends of a bone cut end, said bone piece osteoplastically extending a callus from said bone cut end and movably arranged along a lengthwise direction of said plate; and
a wire connected to said bracket so that said bracket moves in the lengthwise direction of said plate so as to osteoplastically extend said callus.

2. A callus elongating/regenerative device comprising:
a plate provided in a bar-shaped fashion to move in a three-dimensional direction depending on a pre-mutilated bone shape so as to bridge a mutilated bone portion;
a bracket having a slide portion movably provided while surrounding said plate, said bracket holding a bone piece located at said mutilated bone portion formed between both ends of a bone cut end, said bone piece osteoplastically extending a callus from said bone cut end and movably arranged along a lengthwise direction of said plate, said bone cut end being severed to produce said bone piece; and
a wire connected to said bracket so that said bracket moves in the lengthwise direction of said plate so as to osteoplastically extend a callus formed between a severed end surface of said bone cut end and a severed end surface of said bone piece, each surface of which appeared when said bone cut end is severed.

3. A callus elongating/regenerative device according to claim 1 or 2, wherein a traction mechanism is provided to pull said wire by a tractive force.

4. A callus elongating/regenerative device according to claim 1 or 2, wherein a plurality of said brackets and said wires are provided and each number of said brackets and said wires is two, whereby said callus is osteoplastically extended from two said bone cut ends with said bone pieces held by said brackets.

5. A callus elongating/regenerative device according to claim 4, wherein one of said two brackets has a passage hole to pass said wire therethrough to move the other bracket, and said other bracket has a passage hole to pass said wire therethrough to move said one bracket.

6. A callus elongating/regenerative device according to claim 1 or 2, wherein said plate has a groove to accommodate said wire in the lengthwise direction of said plate.

7. A callus elongating/regenerative device according to claim 3, wherein said traction mechanism comprising:
a rod connected to said wire;
a cylindrical sleeve provided to movably hold said rod in its axial direction with said sleeve fixedly positioned against said plate; and
a stress concentration portion provided with said sleeve to concentrate a bending moment on said sleeve when applied to said sleeve.

8. A callus elongating/regenerative device according to claim 3, wherein said traction mechanism comprising:
a rod connected to said wire;
a cylindrical sleeve provided to movably hold said rod in its axial direction with said sleeve fixedly positioned against said plate; and
a graduated portion provided with said rod to indicate its lengthwise dimension.

9. A callus elongating/regenerative device according to claim 3, wherein said traction mechanism comprising:
a rod connected to said wire;
a cylindrical sleeve provided to movably hold said rod in its axial direction with said sleeve fixedly positioned against said plate; and
a tension measuring member provided to measure a tension applied to said wire when said bracket is pulled by the tractive force.

10. A callus elongating/regenerative device according to claim 3, wherein said traction mechanism comprising:
a rod connected to said wire, and having a male-threaded portion on an outer surface of said rod;
a cylindrical sleeve provided to movably hold said rod in its axial direction with said sleeve fixedly positioned against said plate; and
an adjustment member having a female-threaded portion into which said male-threaded portion is screwed, said female-threaded portion being driven to move along said rod so as to adjust a displacement of said bracket pulled by said wire.

11. A callus elongating/regenerative device according to claim 10, wherein said wire is held at a hollow space appeared between an inner wall of said sleeve and an outer wall of said rod, and said wire removably engaging with an end surface of said rod.
